(19) United States
(12) Patent Application Publication
(10) Pub. No.: US 2024/0285157 A1
(43) Pub. Date: Aug. 29, 2024

(54) MEDICAL OBSERVATION SYSTEM, INFORMATION PROCESSING APPARATUS, AND INFORMATION PROCESSING METHOD

(71) Applicant: SONY GROUP CORPORATION, TOKYO (JP)

(72) Inventors: DAISUKE NAGAO, TOKYO (JP); KENJI TAKAHASHI, TOKYO (JP); KEI TOMATSU, TOKYO (JP); YOHEI KURODA, TOKYO (JP); MASAYUKI UMEJIMA, TOKYO (JP)

(21) Appl. No.: 18/573,748

(22) PCT Filed: Feb. 14, 2022
(86) PCT No.: PCT/JP2022/005559

§ 371 Date: Dec. 22, 2023

(30) Foreign Application Priority Data
Jun. 30, 2021 (JP) 2021-108329

(51) Int. Cl. A61B 1/06; A61B 1/00

(52) U.S. Cl. CPC A61B 1/0646; A61B 1/00006; A61B 1/000095; A61B 1/00013; A61B 1/00045; A61B 1/00149

(57) ABSTRACT

Real-time performance in image recognition is improved, and a deterioration in robustness is inhibited. A medical observation system according to an embodiment includes: a first imaging unit (11) that includes a plurality of pixels (110) each of which detects a change in luminance of incident light as an event and that acquires an image of an environment in an abdominal cavity of a living body; a polarizing filter (15) disposed on an optical path of the incident light incident on the first imaging unit; and an adjustment unit (20) that adjusts luminance of the incident light incident on the first imaging unit by adjusting luminance of light transmitted through the polarizing filter based on the image acquired by the first imaging unit.

### FIELD

The present disclosure relates to a medical observation system, an information processing apparatus, and an information processing method.

### BACKGROUND

In recent years, developments of artificial intelligence (AI) and robotics have raised expectations for real-time assistance and procedure support with a robot during surgery.

### CITATION LIST

### Patent Literature

Patent Literature 1: JP 2021-020822 A

### SUMMARY

### Technical Problem

For real-time assistance and procedure support using a robot during surgery, it is necessary to robustly measure/recognize an abdominal cavity environment from an image captured by using an endoscope, for example.

Here, illuminating the abdominal cavity is necessary in laparoscopic surgery. There are objects having high reflectance such as body fluid in the abdominal cavity. Light reflected by these objects has higher intensity than light reflected by another organ and the like, which may generate so-called blown out highlights in which a pixel value is saturated. This causes a deterioration in robustness in image recognition.

Thus, the present disclosure proposes a medical observation system, an information processing apparatus, and an information processing method capable of inhibiting the deterioration in robustness in image recognition.

### Solution to Problem

To solve the problems described above, a medical observation system according to an embodiment of the present disclosure includes: a first imaging unit that includes a plurality of pixels each of which detects a change in luminance of incident light as an event and that acquires an image of an environment in an abdominal cavity of a living body; a polarizing filter disposed on an optical path of the incident light incident on the first imaging unit; and an adjustment unit that adjusts luminance of the incident light incident on the first imaging unit by adjusting luminance of light transmitted through the polarizing filter based on the image acquired by the first imaging unit.

### BRIEF DESCRIPTION OF DRAWINGS

FIG. 1 illustrates one example of a schematic configuration of an endoscopic surgery system according to a first embodiment.

FIG. 2 is a schematic diagram illustrating a configuration example of an endoscope according to the first embodiment.

FIG. 3 is a block diagram illustrating an example of a schematic configuration of an image sensor according to the first embodiment.

FIG. 4 is a block diagram illustrating an example of a schematic configuration of an EVS according to the first embodiment.

FIG. 5 is a block diagram illustrating one example of a functional block configuration of the endoscopic surgery system according to the first embodiment.

FIG. 6 is a flowchart illustrating one example of a main operation of the endoscopic surgery system according to the first embodiment.

FIG. 7 is a flowchart illustrating one example of a polarizing filter adjustment operation according to the first embodiment.

FIG. 8 is a graph illustrating one example of the relation between a filter angle and luminance constructed in Step S115 of FIG. 7.

FIG. 9 is a flowchart illustrating a first variation of the polarizing filter adjustment operation according to the first embodiment.

FIG. 10 is a flowchart illustrating a second variation of the polarizing filter adjustment operation according to the first embodiment.

FIG. 11 is a schematic diagram illustrating a configuration example of a first variation of an adjustment mechanism according to the first embodiment.

FIG. 12 is a schematic diagram illustrating another configuration of a second variation of the adjustment mechanism according to the first embodiment.

FIG. 13 is a schematic diagram illustrating another configuration of a third variation of the adjustment mechanism according to the first embodiment.

FIG. 14 is a schematic diagram illustrating one example of a drive unit that moves an EVS according to a second embodiment.

FIG. 15 is a hardware configuration diagram illustrating one example of a computer that implements a function of an information processing apparatus according to the present disclosure.

### DESCRIPTION OF EMBODIMENTS

Embodiments of the present disclosure will be described in detail below with reference to the drawings. Note that, in the following embodiments, the same reference signs are attached to the same parts to omit duplicate description.

Furthermore, the present disclosure will be described in accordance with the following item order.

### 1. First Embodiment

1.1 Schematic Configuration of Endoscopic Surgery System 5000
1.2 Example of Detailed Configuration of Support Arm Portion 5027
1.3 Example of Detailed Configuration of Light Source Device 5043
1.4 Configuration Example of Endoscope 5001
1.5 Configuration Example of Image Sensor
1.6 Configuration Example of EVS
1.7 Functional Block Configuration Example
1.8 Operation Example
1.8.1 Main Operation Example
1.8.2 Example of Polarizing Filter Adjustment Operation
1.9 Actions/Effects
1.10 Variations
1.10.1 Variations of Polarizing Filter Adjustment Operation
1.10.1.1 First Variation
1.10.1.2 Second Variation
1.10.2 Variations of Adjustment Mechanism
1.10.2.1 First Variation
1.10.2.2 Second Variation
1.10.2.3 Third Variation
2. Second Embodiment
3. Hardware Configuration

### 1. First Embodiment

A medical observation system, an information processing apparatus, and an information processing method according to a first embodiment of the present disclosure will be described in detail below with reference to the drawings. Although an endoscopic surgery system is exemplified as the medical observation system in the following description, the medical observation system is not limited to the endoscopic surgery system. A technique according to the embodiment may be applied to various systems that execute predetermined processing on an image obtained by imaging space with low brightness.

### 1.1 Schematic Configuration of Endoscopic Surgery System 5000

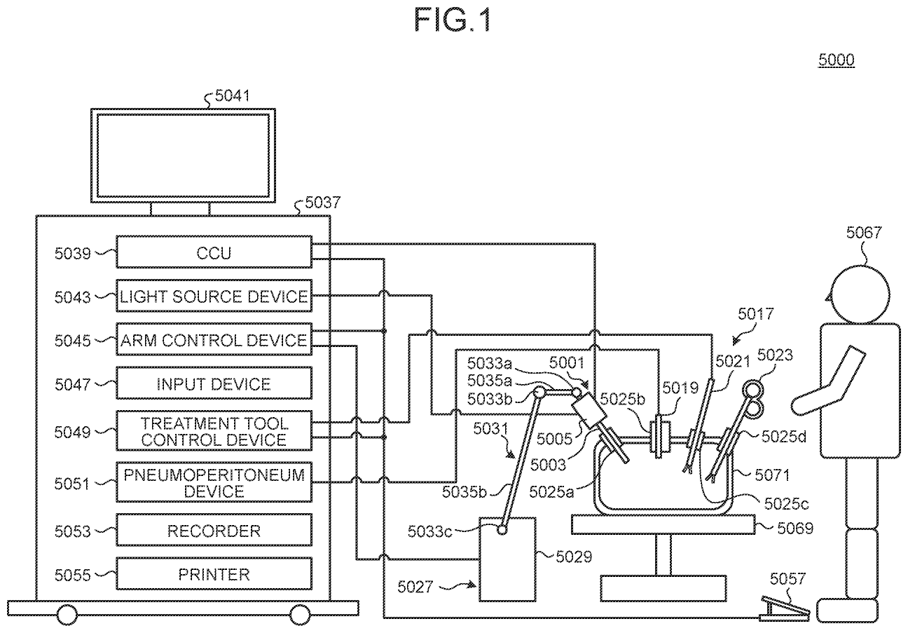

First, a schematic configuration of an endoscopic surgery system 5000 according to the embodiment will be described. FIG. 1 illustrates one example of the schematic configuration of the endoscopic surgery system according to the embodiment. FIG. 1 illustrates how a surgeon 5067 performs surgery on a patient 5071 on a patient bed 5069 by using the endoscopic surgery system 5000. As illustrated in FIG. 1, the endoscopic surgery system 5000 includes an endoscope 5001, other surgical tools 5017, a support arm portion 5027, and a cart 5037. The support arm portion 5027 supports the endoscope 5001. Various devices for endoscopic surgery are mounted in the cart 5037. Details of the endoscopic surgery system 5000 will be sequentially described below.

### (Surgical Tools 5017)

In endoscopic surgery, instead of cutting the abdominal wall and opening the abdomen, the abdominal wall is punctured with a plurality of cylindrical hole making instruments called trocars 5025a to 5025d, for example. Then, a lens barrel 5003 of the endoscope 5001 and the other surgical tools 5017 are inserted from the trocars 5025a to 5025d into the body cavity of the patient 5071. In an example of FIG. 1, a pneumoperitoneum tube 5019, an energy treatment tool 5021, and forceps 5023 are inserted into the body cavity of the patient 5071 as the other surgical tools 5017. Furthermore, the energy treatment tool 5021 is used to incise and peel off a tissue, seal a blood vessel, and the like by high-frequency current and ultrasonic vibration. Note, however, that the surgical tools 5017 in FIG. 1 are merely examples, and examples of the surgical tools 5017 include various surgical tools commonly used in endoscopic surgery, such as tweezers and a retractor.

### (Support Arm Portion 5027)

The support arm portion 5027 includes an arm portion 5031 extending from a base portion 5029. In the example of FIG. 1, the arm portion 5031 includes joints 5033a, 5033b, and 5033c and links 5035a and 5035b. The arm portion 5031 is driven under the control of an arm control device 5045. Then, the arm portion 5031 supports the endoscope 5001. The position and posture of the endoscope 5001 are controlled. This enables the position of the endoscope 5001 to be stably fixed.

### (Endoscope 5001)

The endoscope 5001 includes the lens barrel 5003 and a camera head 5005. A region of a predetermined length from a distal end of the lens barrel 5003 is inserted into the body cavity of the patient 5071. The camera head 5005 is connected to a proximal end of the lens barrel 5003. Although, in the example of FIG. 1, the endoscope 5001 configured as a so-called rigid scope including a rigid lens barrel 5003 is illustrated, the endoscope 5001 may be configured as a so-called flexible scope including a flexible lens barrel 5003. In the embodiment of the present disclosure, the endoscope 5001 is not particularly limited to be configured as any thereof.

An opening is provided at a distal end of the lens barrel 5003. An objective lens is fitted into the opening. A light source device (medical light source device) 5043 is connected to the endoscope 5001. Light generated by the light source device 5043 is guided to the distal end of the lens barrel by a light guide extending inside the lens barrel 5003, and applied to an object to be observed in the body cavity (e.g., abdominal cavity) of the patient 5071 via the objective lens. Note that, in the embodiment of the present disclosure, the endoscope 5001 may be a front direct-view scope or an oblique-view scope, and is not particularly limited.

An optical system and an imaging element are provided inside the camera head 5005. Reflected light (observation light) from the object to be observed is collected on the imaging element by the optical system. The observation light is photoelectrically converted by the imaging element to generate an electric signal corresponding to the observation light, that is, a pixel signal corresponding to an observation image. The pixel signal is transmitted to a camera control unit (CCU) 5039 as raw data. Note that the camera head 5005 has a function of adjusting a magnification and a focal length by appropriately driving the optical system thereof.

Note that a plurality of imaging elements may be provided in the camera head 5005 in order to support, for example, stereoscopic viewing (stereo system). In this case, a plurality of relay optical systems is provided inside the lens barrel 5003 in order to guide observation light to each of the plurality of imaging elements. Furthermore, in the embodiment of the present disclosure, different types of imaging elements can be provided, which will be described later. Moreover, details of the camera head 5005 and the lens barrel 5003 according to the embodiment of the present disclosure will also be described later.

### (Various Devices Mounted in Cart)

First, a display device 5041 displays an image based on a pixel signal subjected to image processing by the CCU 5039 under the control of the CCU 5039. When the endoscope 5001 supports imaging of high resolution such as 4K (number of horizontal pixels: 3840×number of vertical pixels: 2160) and 8K (number of horizontal pixels: 7680×number of vertical pixels: 4320) and/or 3D display, a display device capable of performing high-resolution display corresponding to each thereof and/or 3D display is used as the display device 5041. Furthermore, a plurality of display devices 5041 having different resolutions and sizes may be provided depending on uses.

Furthermore, an image of a surgical site in the body cavity of the patient 5071 captured by the endoscope 5001 is displayed on the display device 5041. While looking at the image of the surgical site displayed on the display device 5041 in real time, the surgeon 5067 can perform treatment such as resection of an affected part by using the energy treatment tool 5021 and the forceps 5023. Note that, although not illustrated, the surgeon 5067, an assistant, and the like may support the pneumoperitoneum tube 5019, the energy treatment tool 5021, and the forceps 5023 during surgery.

Furthermore, the CCU 5039 includes a central processing unit (CPU) and a graphics processing unit (GPU). The CCU 5039 can integrally controls operations of the endoscope 5001 and the display device 5041. Specifically, the CCU 5039 performs various pieces of image processing for displaying an image based on a pixel signal received from the camera head 5005, such as development processing (demosaic processing), on the pixel signal. Moreover, the CCU 5039 provides the pixel signal subjected to the image processing to the display device 5041. Furthermore, the CCU 5039 transmits a control signal to the camera head 5005, and controls driving thereof. The control signal can include information on imaging conditions such as a magnification and a focal length. Note that details of the CCU 5039 according to the embodiment of the present disclosure will be described later.

The light source device 5043 includes a light source such as a light emitting diode (LED). The light source device 5043 supplies irradiation light to be used at the time when the surgical site is imaged to the endoscope 5001. Note that details of the light source device 5043 according to the embodiment of the present disclosure will be described later.

The arm control device 5045 includes, for example, a processor such as a CPU. The arm control device 5045 operates in accordance with a predetermined program to control driving of the arm portion 5031 of the support arm portion 5027 in accordance with a predetermined control method. Note that details of the arm control device 5045 according to the embodiment of the present disclosure will be described later.

An input device 5047 is an input interface for the endoscopic surgery system 5000. The surgeon 5067 can input various pieces of information and instructions to the endoscopic surgery system 5000 via the input device 5047. For example, the surgeon 5067 inputs various pieces of information on surgery, such as body information on a patient and information on a surgical procedure of the surgery, via the input device 5047. Furthermore, for example, the surgeon 5067 can input an instruction to drive the arm portion 5031, an instruction to change an imaging condition (e.g., type of irradiation light, magnification, and focal length) of the endoscope 5001, and an instruction to drive the energy treatment tool 5021 via the input device 5047. Note that the type of the input device 5047 is not limited, and the input device 5047 may be various known input devices. For example, a mouse, a keyboard, a touch panel, a switch, a foot switch 5057, and/or a lever can be applied as the input device 5047. For example, when a touch panel is used as the input device 5047, the touch panel may be provided on a display surface of the display device 5041.

Alternatively, the input device 5047 may be a device worn on a part of the body of the surgeon 5067, such as a glasses-type wearable device and a head mounted display (HMD). In this case, various inputs are given in accordance with a gesture and a line of sight of the surgeon 5067 detected by these devices. Furthermore, the input device 5047 can include a camera capable of detecting movement of the surgeon 5067. Various inputs may be given in accordance with the gesture and the line of sight of the surgeon 5067 detected from an image captured by the camera. Moreover, the input device 5047 can include a microphone capable of collecting voice of the surgeon 5067. Various inputs may be given by voice via the microphone. The input device 5047 is configured to be able to input various pieces of information in a non-contact manner as described above, so that, in particular, a user (e.g., surgeon 5067) belonging to a clean area can operate a device belonging to an unclean area in a non-contact manner. Furthermore, the surgeon 5067 can operate the device without releasing his/her hand from a surgical tool which the surgeon 5067 holds, which improves convenience for the surgeon 5067.

A treatment tool control device 5049 controls driving of the energy treatment tool 5021 for performing cautery and incision of a tissue or sealing of a blood vessel. A pneumoperitoneum device 5051 feeds gas into the body cavity of the patient 5071 through the pneumoperitoneum tube 5019 to inflate the body cavity in order to secure a field of view of the endoscope 5001 and secure workspace for the surgeon 5067. A recorder 5053 is a device capable of recording various pieces of information on surgery. A printer 5055 is a device capable of printing various pieces of information on surgery in various forms such as text, an image, and a graph.

### 1.2 Example of Detailed Configuration of Support Arm Portion 5027

Moreover, one example of a detailed configuration of the support arm portion 5027 will be described. The support arm portion 5027 includes a base portion 5029 and the arm portion 5031. The base portion 5029 is a base. The arm portion 5031 extends from the base portion 5029. In the example of FIG. 1, the arm portion 5031 includes a plurality of joints 5033a, 5033b, and 5033c and a plurality of links 5035a and 5035b connected by the joint 5033b. For the sake of simplicity, FIG. 1 illustrates a simplified configuration of the arm portion 5031. Specifically, the shapes, numbers, and arrangements of the joints 5033a to 5033c and the links 5035a and 5035b, the directions of rotation axes of the joints 5033a to 5033c, and the like can be appropriately set such that the arm portion 5031 has a desired degree of freedom. For example, the arm portion 5031 can preferably have a degree of freedom of six or more. This enables the endoscope 5001 to freely move within a movable range of the arm portion 5031, so that the lens barrel 5003 of the endoscope 5001 can be inserted into the body cavity of the patient 5071 from a desired direction.

Actuators are provided in the joints 5033a to 5033c. The joints 5033a to 5033c can rotate around predetermined rotation axes by driving of the actuators. The arm control device 5045 controls the driving of the actuators to control a rotation angle of each of the joints 5033a to 5033c and control driving of the arm portion 5031. This can achieve controls of the position and posture of the endoscope 5001. In the case, the arm control device 5045 can control the driving of the arm portion 5031 by various known control methods such as force control and position control.

For example, the position and posture of the endoscope 5001 may be controlled by the surgeon 5067 appropriately giving an operation input via the input device 5047 (including foot switch 5057) and thereby the driving of the arm portion 5031 being appropriately controlled by the arm control device 5045 in accordance with the operation input. Note that the arm portion 5031 may be operated by a so-called master-slave method. In this case, the arm portion 5031 (slave) can be remotely operated by the surgeon 5067 via the input device 5047 (master console) installed at a place away from a surgery room or in the surgery room.

Here, doctors called scopists have commonly supported the endoscope 5001 in endoscopic surgeries. In contrast, in the embodiment of the present disclosure, using the support arm portion 5027 enables the position of the endoscope 5001 to be reliably fixed without a human hand, so that an image of a surgical site can be stably obtained, and surgery can be smoothly performed.

Note that the arm control device 5045 is not necessarily provided in the cart 5037. Furthermore, the arm control device 5045 is not necessarily one device. For example, the arm control device 5045 may be provided in each of the joints 5033a to 5033c of the arm portion 5031 of the support arm portion 5027. Driving of the arm portion 5031 may be controlled by a plurality of arm control devices 5045 cooperating with each other.

### 1.3 Example of Detailed Configuration of Light Source Device 5043

Next, one example of a detailed configuration of the light source device 5043 will be described. The light source device 5043 supplies irradiation light for imaging a surgical site to the endoscope 5001. The light source device 5043 includes a white light source including, for example, an LED, a laser light source, or a combination thereof. In this case, when a combination of RGB laser light sources constitutes the white light source, output intensity and output timing of each color (each wavelength) can be controlled with high accuracy, so that the light source device 5043 can adjust the white balance of a captured image. Furthermore, in this case, an object to be observed is irradiated in time division with laser light from each of the RGB laser light sources, and driving of an imaging element of the camera head 5005 is controlled in synchronization with the irradiation timing, so that an image corresponding to each of RGB can be captured in time division. According to the method, a color image can be obtained without providing a color filter in the imaging element.

Furthermore, driving of the light source device 5043 may be controlled such that the intensity of light to be output is change for each predetermined time. An image of a high dynamic range without so-called blocked-up shadows and blown out highlights can be generated by acquiring images in time division controlling driving of the imaging element of the camera head 5005 in synchronization with timing of change in the intensity of the light and combining the images.

Furthermore, the light source device 5043 may be able to supply light in a predetermined wavelength band supporting special light observation. In the special light observation, light in a band narrower than that of irradiation light (i.e., white light) at the time of usual observation is applied by using wavelength dependency of light absorption in a body tissue, whereby so-called narrow band imaging is performed. In the narrow band imaging, a predetermined tissue such as a blood vessel in a superficial portion of the mucous membrane is imaged with high contrast. Alternatively, in the special light observation, fluorescent observation may be performed. In the fluorescent observation, an image is obtained by fluorescence generated by applying excitation light. In the fluorescent observation, excitation light may be applied to a body tissue, and fluorescence from the body tissue may be observed (autofluorescence observation). Alternatively, a fluorescent image may be obtained by locally injecting a reagent such as indocyanine green (ICG) to the body tissue and applying excitation light in accordance with the fluorescence wavelength of the reagent to the body tissue. The light source device 5043 can supply narrow-band light and/or excitation light supporting such special light observation.

### 1.4 Configuration Example of Endoscope 5001

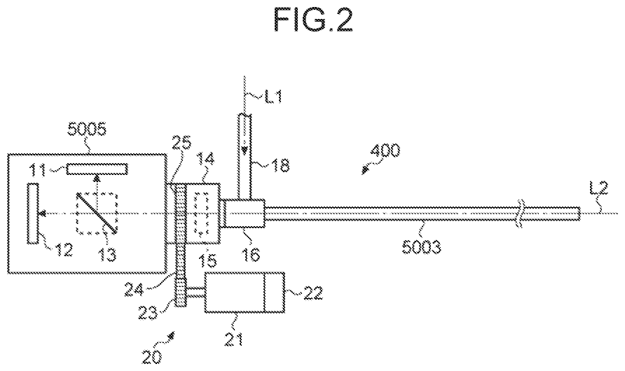

Next, a configuration example of the endoscope 5001 according to the embodiment will be described with reference to FIG. 2. FIG. 2 is a schematic diagram illustrating the configuration example of the endoscope according to the embodiment. As illustrated in FIG. 2, the endoscope 5001 includes, for example, the camera head 5005 and an optical system 400.

### (Camera Head 5005)

An image sensor 11, an event-based vision sensor (EVS) 12, and a beam splitter 13 are provided inside the camera head 5005. The image sensor 11 generates RGB image data. The EVS 12 generates event data. For example, when the light source device 5043 outputs light having a wavelength outside a visible light region (e.g., infrared light and near-infrared light) in addition to visible light, an optical element that splits incident light in accordance with the wavelength thereof, such as a prism and a dichroic mirror, may be used as the beam splitter 13. In contrast, when the light source device 5043 outputs only visible light, a half mirror or the like may be used as the beam splitter 13.

In the camera head 5005, the image sensor 11 and the EVS 12 are arranged such that planes including light receiving surfaces thereof are substantially perpendicular to each other, for example. In the example of FIG. 2, light that has passed through the beam splitter 13 is incident on the image sensor 11, and light reflected by the beam splitter 13 is incident on the EVS 12. That is, in the embodiment, the image sensor 11 and the EVS 12 share the same optical axis. In the case, a coordinate system of the RGB image data acquired by the image sensor 11 may be aligned with a coordinate system of event image data acquired by the EVS 12 by aligning an angle of view of the image sensor 11 with an angle of view of the EVS 12.

### (Optical System 400)

The optical system 400 includes the above-described lens barrel 5003, a joint portion 14, and a merging portion 16.

The lens barrel 5003 has a configuration in which an optical fiber is passed through a cylindrical barrel made of metal such as stainless steel. A lens is provided at a distal end of the lens barrel 5003. A rear end of the lens barrel 5003 is fixed to the joint portion 14 via the merging portion 16.

An optical fiber cable 18 is inserted into the merging portion 16 from the side. The optical fiber cable 18 guides irradiation light L1 output from the light source device 5043. The optical fiber cable 18 passes through the lens barrel 5003 from the merging portion 16, and is guided to the distal end of the lens barrel 5003. Therefore, the irradiation light L1 output from the light source device 5043 is emitted from the distal end of the lens barrel 5003 via the optical fiber cable 18.

The joint portion 14 is detachable from the camera head 5005, for example. Light L2 (reflected light of irradiation light L1) incident on the distal end of the lens barrel 5003 is propagated through an optical fiber (note, however, that optical fiber different from the optical fiber cable 18) in the lens barrel 5003, and guided to the inside of the camera head 5005.

Note that the optical system 400 may include one or more lenses such as a zoom lens and a focus lens. In that case, the optical system 400 may further include a mechanism for moving the positions of the zoom lens and the focus lens along an optical axis in order to, for example, adjust the magnification and focus of a captured image (RGB image data and event image data).

Furthermore, in the embodiment, the optical system 400 further includes a polarizing filter 15 for limiting the polarization direction of the reflected light L2 incident on the camera head 5005. The polarizing filter 15 may be various linear polarizers that selectively transmit a linearly polarized component in a specific direction of incident light, such as a birefringent polarizer, a linear dichroic polarizer, and a Brewster polarizer.

Moreover, the optical system 400 includes an adjustment mechanism 20 that adjusts the polarization direction of the reflected light L2 incident on the camera head 5005. More specifically, the optical system 400 includes the adjustment mechanism 20 for adjusting the polarization direction of the polarizing filter 15 around the optical axis of the reflected light L2. The adjustment mechanism 20 may include, for example, a drive unit 21, a gear 23, a gear 25, and a gear 24. The drive unit 21 includes a motor. The gear 23 is fixed to a rotation axis of the drive unit 21. The gear 25 is provided on a part or the whole of a side surface of the joint portion 14. The gear 24 transmits rotation of the gear 23 to the gear 25. In that case, the polarizing filter 15 may be fixed to the inside of the joint portion 14 rotatably attached to the camera head 5005, for example. Note, however, that this is not a limitation. Various changes may be made to have configurations in which the drive unit 21 may be arranged inside the joint portion 14 and the polarizing filter 15 may be directly and rotationally moved. Furthermore, an encoder 22 (and potentiometer and the like) for detecting a rotation angle around the optical axis of the polarizing filter 15 may be provided in the adjustment mechanism 20.

As described above, an amount of light reflected by an object having high reflectance such as body fluid can be reduced by narrowing the reflected light L2 incident on the camera head 5005, that is, the image sensor 11 and the EVS 12 in the camera head 5005 down to a linearly polarized component in an adjusted specific direction, which can inhibit occurrence of blown out highlights and the like in the RGB image data and/or the event image data. Light reflected by an object having high reflectance such as body fluid has a large amount of linearly polarized components in the polarization direction in accordance with a reflection surface of the object. The amount of the light reflected by an object having high reflectance such as body fluid can be effectively reduced while a decrease in an amount of light reflected by another object such as an organ is inhibited by using the polarizing filter 15 capable of narrowing incident light down to a linearly polarized component in the specific direction.

Note that, in the embodiment, the camera head 5005, the optical system 400, and the like described above may have sealed structure having high airtightness and waterproofness in order to have resistance to autoclave sterilization processing.

Furthermore, in the embodiment, the image sensor 11 may include a pair of image sensors that acquire an image for a right eye and an image for a left eye for supporting 3D display (stereo system). The 3D display enables the surgeon 5067 to accurately grasp the depth of a living body tissue (organ) in a surgical site and grasp a distance to the living body tissue.

Furthermore, the beam splitter 13 may have a function of adjusting the distribution ratio between an amount of light incident on the EVS 12 and an amount of light incident on the image sensor 11. For example, the above-described function can be provided by adjusting the transmittance of beam splitter 13. More specifically, for example, when the optical axis of the reflected light L2 is the same between the EVS 12 and the image sensor 11, the transmittance of the beam splitter 13 may be adjusted such that an amount of light incident on the side of the image sensor 11 increases.

A high dynamic range, high robustness in detecting a subject moving fast, and high time resolution, which are features of the EVS 12, and high tracking performance for a long time, which is a feature of the image sensor 11, can be utilized by combining the EVS 12 with the image sensor 11 as described above, so that accuracy of recognizing the subject can be improved.

Note, however, that the embodiment is not limited to the configuration in which the reflected light L2 is guided to both the EVS 12 and the image sensor 11 by the beam splitter 13. For example, a hybrid-type sensor in which pixel arrays corresponding to the EVS 12 and the image sensor 11 are provided on the same substrate (light receiving surface) may be used. In such a case, the above-described beam splitter 13 can be omitted, so that the configuration of the inside of the camera head 5005 can be simplified.

Furthermore, in the embodiment, the camera head 5005 may include an IR sensor (not illustrated) that detects infrared light.

Furthermore, in the embodiment of the present disclosure, in order to achieve a stereo system capable of measuring a distance, two EVSs 12 and two image sensors 11 may be provided, or three or more of EVSs 12 and three or more of image sensors 11 may be provided. Furthermore, when the stereo system is achieved, two image circles may be projected on one pixel array by causing two optical systems 400 to support one pixel array.

Furthermore, in the embodiment, the EVS 12 and the image sensor 11 may be provided in distal end portions of a flexible scope and a rigid scope inserted into the abdominal cavity.

### 1.5 Configuration Example of Image Sensor

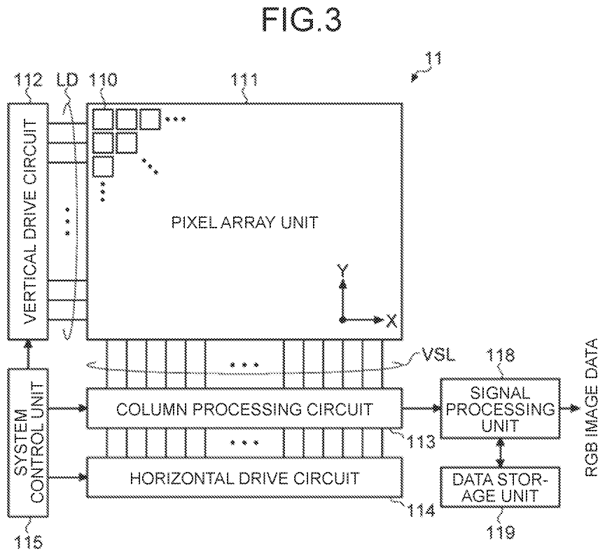

FIG. 3 is a block diagram illustrating an example of a schematic configuration of the image sensor according to the first embodiment. Note that, although, in the example, a complementary metal-oxide-semiconductor (CMOS) type image sensor is exemplified, this is not a limitation. Various image sensors capable of acquiring color or monochrome image data, such as a charge-coupled device (CCD) type image sensor, may be used. Furthermore, the CMOS type image sensor may be created by applying or partially using a CMOS process.

As illustrated in FIG. 3, the image sensor 11 has, for example, a stack structure in which a semiconductor chip having a pixel array unit 111 and a semiconductor chip having a peripheral circuit are stacked. The peripheral circuit may include, for example, a vertical drive circuit 112, a column processing circuit 113, a horizontal drive circuit 114, and a system control unit 115.

The image sensor 11 further includes a signal processing unit 118 and a data storage unit 119. The signal processing unit 118 and the data storage unit 119 may be provided on the same semiconductor chip as the peripheral circuit, or may be provided on another semiconductor chip.

The pixel array unit 111 has a configuration in which pixels 110 are arranged in a row direction and a column direction, that is, in a two-dimensional lattice shape in a matrix. The pixels 110 include photoelectric conversion elements that generate and accumulate charges in accordance with an amount of received light. Here, the row direction refers to a direction of arrangement of pixels in a pixel row (horizontal direction in figure). The column direction refers to a direction of arrangement of pixels in a pixel column (vertical direction in figure).

In the pixel array unit 111, for a pixel arrangement in a matrix, pixel drive lines LD are arranged along the row direction for respective pixel rows, and vertical signal lines VSL are arranged along the column direction for respective pixel columns. A pixel drive line LD transmits a drive signal for performing driving at the time when a signal is read from a pixel. Although FIG. 3 illustrates the pixel drive lines LD as each having one wire, the pixel drive lines LD are not limited to having one wire. One end of the pixel drive line LD is connected to an output end in each row of the vertical drive circuit 112.

The vertical drive circuit 112 includes a shift register and an address decoder. The vertical drive circuit 112 drives pixels of the pixel array unit 111 at the same time for all pixels or in units of rows. That is, the vertical drive circuit 112 includes a drive unit that controls the operation of each pixel of the pixel array unit 111 together with the system control unit 115 that controls the vertical drive circuit 112. Although a specific configuration of the vertical drive circuit 112 is not illustrated, the vertical drive circuit 112 commonly includes two scanning systems of a readout scanning system and a sweep-out scanning system.

The readout scanning system sequentially selects and scans the pixels 110 of the pixel array unit 111 in units of rows in order to read out signals from the pixels 110. The signals read out from the pixels 110 are analog signals. The sweep-out scanning system performs sweep-out scanning on a readout row on which readout scanning is to be performed by the readout scanning system prior to the readout scanning by an exposure time.

Unnecessary charges are swept out from a photoelectric conversion element of a pixel 110 in a readout row by the sweep-out scanning of the sweep-out scanning system, which resets the photoelectric conversion element. Then, a so-called electronic shutter operation is performed by sweeping out (resetting) unnecessary charges with the sweep-out scanning system. Here, the electronic shutter operation refers to an operation of discarding charges of a photoelectric conversion element and newly starting exposure (starting accumulation of charges).

A signal read out by a readout operation of the readout scanning system corresponds to an amount of light received after the last readout operation or electronic shutter operation. Then, a period from readout timing of the last readout operation or sweep-out timing of the electronic shutter operation to readout timing of a readout operation of this time corresponds to a period of accumulation of charges (also referred to as period of exposure) in the pixel 110.

A signal output from each pixel 110 of a pixel row selectively scanned by the vertical drive circuit 112 is input to the column processing circuit 113 through each of the vertical signal lines VSL for each pixel column. The column processing circuit 113 performs predetermined signal processing on a signal output from each pixel of a selected row through a vertical signal line VSL for each pixel column of the pixel array unit 111, and temporarily holds the pixel signal after the signal processing.

Specifically, the column processing circuit 113 performs at least noise removal processing, for example, correlated double sampling (CDS) processing and double data sampling (DDS) processing, as the signal processing. For example, fixed pattern noise unique to a pixel, such as reset noise and threshold variation of an amplification transistor in a pixel, is removed by the CDS processing. In addition, the column processing circuit 113 also has, for example, an analog-digital (AD) conversion function. The column processing circuit 113 converts an analog pixel signal read out from the photoelectric conversion element into a digital signal, and outputs the digital signal.

The horizontal drive circuit 114 includes a shift register and an address decoder. The horizontal drive circuit 114 sequentially selects readout circuits (hereinafter, referred to as pixel circuits) for pixel columns of the column processing circuit 113. Pixel signals subjected to signal processing for each pixel circuit in the column processing circuit 113 are sequentially output by the selective scanning of the horizontal drive circuit 114.

The system control unit 115 includes a timing generator that generates various timing signals. The system control unit 115 controls driving of the vertical drive circuit 112, the column processing circuit 113, and the horizontal drive circuit 114 based on the various timings generated by the timing generator.

The signal processing unit 118 has at least an arithmetic processing function. The signal processing unit 118 performs various pieces of signal processing, such as arithmetic processing, on a pixel signal output from the column processing circuit 113. The data storage unit 119 temporarily stores data necessary for signal processing in the signal processing unit 118.

Note that the RGB image data output from the signal processing unit 118 may be directly input to and displayed on a display device 30 as described above, or may be input to and displayed on the display device 30 after being once input to the CCU 5039 and subjected to predetermined processing.

### 1.6 Configuration Example of EVS

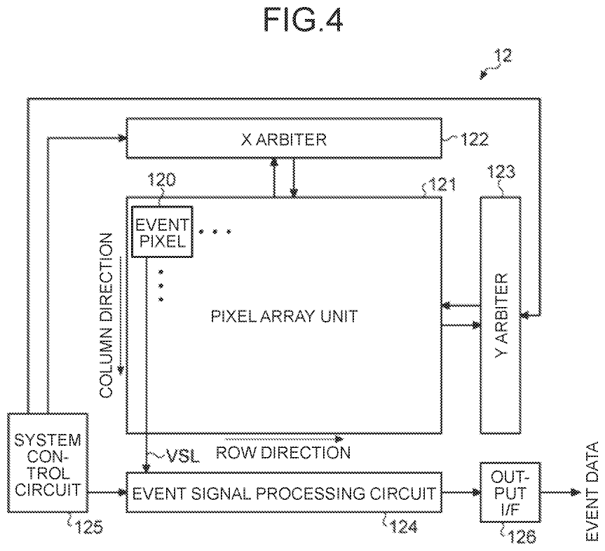

Subsequently, an example of a schematic configuration of the EVS 12 will be described. FIG. 4 is a block diagram illustrating the example of the schematic configuration of the EVS according to the embodiment. As illustrated in FIG. 4, the EVS 12 includes a pixel array unit 121, an X arbiter 122, a Y arbiter 123, an event signal processing circuit 124, a system control circuit 125, and an output interface (I/F) 126.

The pixel array unit 121 has a configuration in which a plurality of event pixels 120 is arranged in a two-dimensional lattice shape. Each of the event pixels 120 detects an event based on a change in luminance of incident light. Note that, in the following description, a row direction refers to a direction of arrangement of pixels in a pixel row (horizontal direction in figure). The column direction refers to a direction of arrangement of pixels in a pixel column (vertical direction in figure).

Each event pixel 120 includes a photoelectric conversion element that generates a charge in accordance with the luminance of incident light. When a change in luminance of the incident light is detected based on photocurrent flowing out from the photoelectric conversion element, the event pixel 120 outputs a request for reading from the event pixel 120 itself to the X arbiter 122 and the Y arbiter 123, and outputs an event signal indicating that an event has been detected in accordance with arbitrations of the X arbiter 122 and the Y arbiter 123.

Each event pixel 120 detects the presence or absence of an event based on whether or not a change exceeding a predetermined threshold has occurred in the photocurrent in accordance with the luminance of incident light. For example, each event pixel 120 detects that the luminance change exceeds a predetermined threshold (positive event) or falls below the predetermined threshold (negative event) as the event.

When detecting an event, the event pixel 120 outputs a request for permission to output an event signal representing the occurrence of the event to each of the X arbiter 122 and the Y arbiter 123. Then, when receiving a response representing the permission to output an event signal from each of the X arbiter 122 and the Y arbiter 123, the event pixel 120 outputs the event signal to the event signal processing circuit 124.

The X arbiter 122 and the Y arbiter 123 arbitrate a request for output of an event signal supplied from each of the plurality of event pixels 120, and transmit a response based on the arbitration result (permission/non-permission of output of event signal) and a reset signal that resets event detection to the event pixel 120 that has output the request.

The event signal processing circuit 124 executes predetermined signal processing on the event signal input from the event pixel 120 to generate and output event data.

As described above, the change in photocurrent generated in the event pixel 120 can also be regarded as a change in an amount (change in luminance) of light incident on a photoelectric conversion unit of the event pixel 120. Therefore, the event can be said as a change in an amount of light (change in luminance) of the event pixel 120 exceeding a predetermined threshold. The event data representing the occurrence of an event includes at least position information such as coordinates representing the position of the event pixel 120 where a change in an amount of light has occurred as an event. The event data can include the polarity of a change in an amount of light in addition to the position information.

It can be said that pieces of event data in a series output from the event pixel 120 at the timing of occurrence of an event implicitly contains time information representing a relative time of the occurrence of the event as long as an interval between the pieces of event data is maintained just as it used to be when the event has occurred.

Note, however, that, if the interval between pieces of event data is not maintained just as it used to be when the event has occurred due to the pieces of event data being stored in a memory, time information implicitly included in the pieces of event data is lost. Thus, the event signal processing circuit 124 may cause time information representing a relative time where an event has occurred, such as a time stamp, to be included in the pieces of event data before the interval between pieces of event data becomes not maintained just as it used to be when the event has occurred.

### Other Configurations

The system control circuit 125 includes a timing generator that generates various timing signals. The system control circuit 125 controls driving of the X arbiter 122, the Y arbiter 123, the event signal processing circuit 124, and the like based on the various timings generated by the timing generator.

The output I/F 126 sequentially outputs event data output in units of rows from the event signal processing circuit 124 to the CCU 5039 as an event stream. In contrast, the CCU 5039 (e.g., event preprocessing unit 514 or event information processing unit 516 to be described later) accumulates the event data input as an event stream for a predetermined frame period to generate event image data of a predetermined frame rate.

### 1.7 Functional Block Configuration Example

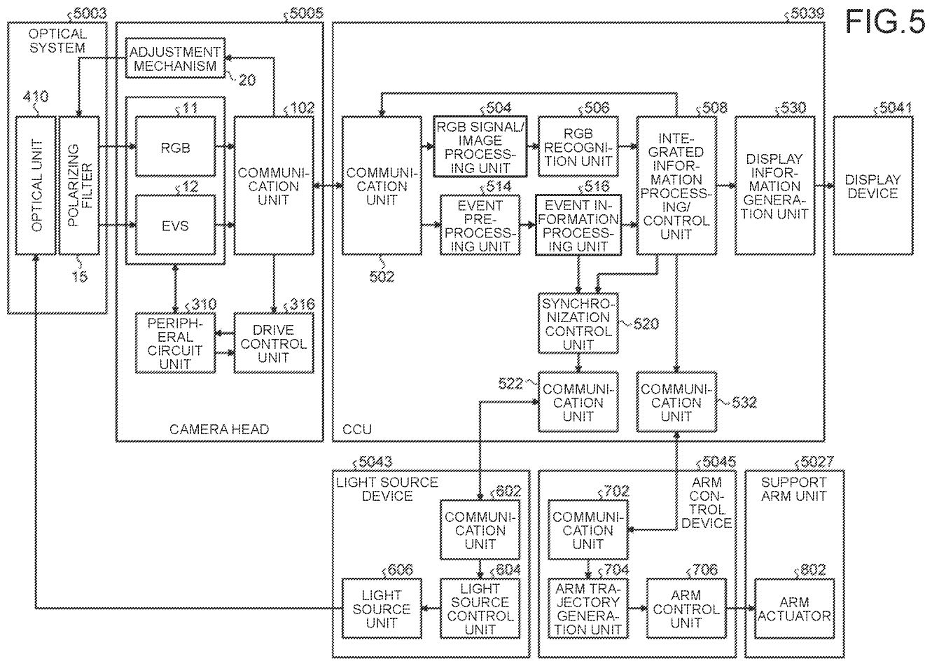

Next, a functional block configuration example of the endoscopic surgery system 5000 according to the embodiment will be described with reference to FIG. 5. FIG. 5 is a block diagram illustrating one example of a functional block configuration of the endoscopic surgery system according to the embodiment. As described above, the endoscopic surgery system 5000 according to the embodiment mainly includes the endoscope 5001, the CCU 5039, the light source device 5043, the arm control device 5045, the support arm portion 5027, and the display device 5041. The endoscope 5001 includes the camera head 5005 and the optical system 400. Then, the camera head 5005, the CCU 5039, the light source device 5043, and the arm control device 5045 can be connected so as to be able to bidirectionally communicate with one another via a transmission cable (not illustrated). Alternatively, the camera head 5005, the CCU 5039, the light source device 5043, and the arm control device 5045 may be connected so as to be able to bidirectionally and wirelessly communicate with one another. As described above, when communication is wirelessly performed, the transmission cable is not required to be provided in a surgery room, which can resolve a situation in which the transmission cable hinders movement of medical staff (e.g., surgeon 5067) in the surgery room. Each device of the endoscopic surgery system 5000 will be described below.

### (Camera Head 5005)

As illustrated in FIG. 5, the camera head 5005 mainly includes the image sensor 11, the EVS 12, a communication unit 102, a peripheral circuit unit 310, a drive control unit 316, and the adjustment mechanism 20. Specifically, the EVS 12 and the image sensor 11 receive the reflected light L2, generates a signal, and outputs the generated signal to the communication unit 102. An optical unit 410 guides the reflected light L2. The optical unit 410 includes an optical fiber and one or more lenses provided in the lens barrel 5003.

The communication unit 102 includes a communication device for transmitting and receiving various pieces of information to and from the CCU 5039. The communication unit 102 can transmit signals from the pixel array unit 121 and the image sensor 11 to the CCU 5039, and receive a control signal for controlling driving of the camera head 5005 from the CCU 5039. The control signal includes information on an imaging condition, such as an information that a frame rate of imaging is designated, information that an exposure value at the time of imaging is designated, and/or information that the magnification and the focus of a captured image are designated. Note that the imaging condition such as a frame rate, an exposure value, a magnification, and a focus may be automatically set by an integrated information processing/control unit 508 of the CCU 5039 based on an acquired image.

The drive control unit 316 controls driving of the EVS 12 based on a control signal from the CCU 5039 received via the communication unit 102. For example, the drive control unit 316 adjusts a threshold and the like to be compared with a luminance change amount at the time when an event is detected.

Furthermore, the reflected light L2 incident on the EVS 12 and the image sensor 11 is limited to a linearly polarized component in a specific polarization direction by the polarizing filter 15 arranged on an optical path. The adjustment mechanism 20 adjusts the polarization direction around the optical axis of the polarizing filter 15. This causes an amount of reflected light L2 (also referred to as luminance or light amount profile) incident on the EVS 12 and the image sensor 11 to be adjusted.

### (CCU 5039)

As illustrated in FIG. 5, the CCU 5039 mainly includes communication units 502, 522, and 532, an RGB signal/image processing unit 504, an RGB recognition unit 506, the integrated information processing/control unit 508, the event preprocessing unit 514, the event information processing unit 516, a synchronization control unit 520, and a display information generation unit 530.

The communication units 502, 522, and 532 include a communication device for transmitting and receiving various pieces of information (e.g., detection signal and control signal) to and from the camera head 5005, the light source device 5043, and the arm control device 5045. In the embodiment, cooperation of the camera head 5005, the light source device 5043, and the support arm portion 5027 is made possible by providing the communication units 502, 522, and 532.

The RGB signal/image processing unit 504 can perform various pieces of image processing on the pixel signal acquired via the communication unit 502, and output the pixel signal to the RGB recognition unit 506 to be described later. The pixel signal is raw data transmitted from the image sensor 11 of the camera head 5005. Examples of the image processing include various pieces of known signal processing such as development processing, image quality improving processing (e.g., band emphasis processing, super-resolution processing, noise reduction (NR) processing, and/or camera shake correction processing), and/or enlargement processing (electronic zoom processing). More specifically, the RGB signal/image processing unit 504 includes a processor such as a CPU and a GPU. The processor operates in accordance with a predetermined program, whereby the above-described image processing is performed. Note that, when the RGB signal/image processing unit 504 includes a plurality of GPUs, the RGB signal/image processing unit 504 may appropriately divide information related to a pixel signal, and perform image processing in parallel with the plurality of GPUS.

The RGB recognition unit 506 can perform recognition processing on an image processed by the RGB signal/image processing unit 504 in order to obtain information for controlling the image sensor 11, the light source device 5043, and the support arm portion 5027. For example, the RGB recognition unit 506 recognizes the position, shape, clearness, luminance, and the like of a subject from an image, and can obtain information for controlling the focus of the image sensor 11, the intensity and range of light applied from the light source device 5043, and driving of the support arm portion 5027. The obtained information is output to the integrated information processing/control unit 508 to be described later. Furthermore, the RGB recognition unit 506 can recognize a surgical tool such as forceps, a specific living body site, bleeding, and the like by segmentation of a subject included in each surgical site image using various image recognition techniques.

The integrated information processing/control unit 508 performs various pieces of processing related to control of the EVS 12 and the image sensor 11 and image display using various pixel signals (first and second outputs) from the EVS 12 and the image sensor 11. For example, the integrated information processing/control unit 508 generates a control signal for controlling the EVS 12 and the image sensor 11. In the case, when the surgeon 5067 inputs an imaging condition, the integrated information processing/control unit 508 may generate the control signal based on the input given by the surgeon 5067. Furthermore, the integrated information processing/control unit 508 can simultaneously perform integration of signals from the EVS 12 and the image sensor 11 and arbitration of images from the EVS 12 and the image sensor 11 having different frame rates. Furthermore, the integrated information

processing/control unit 508 may perform processing such as image quality emphasis, three-dimensional shape measurement, optical flow estimation (e.g., apparent speed of object in image is estimated, or moving object is extracted from image or tracked), visual inertial odometry (posture of camera is estimated and tracked by combination with motion data), motion detection, segmentation, image recognition, and simultaneous localization and mapping (SLAM).

In this way, in the embodiment, the integrated information processing/control unit 508 can perform processing of integrating pieces of output information from the EVS 12 and the image sensor 11, so that an edge of a subject can be clearly captured even in a dark region by output from the EVS 12, for example. Furthermore, the EVS 12 has high time resolution, and can thus complement tracking of a subject of a low frame rate performed by the image sensor 11. In the embodiment, performance of tracking a subject moving or deforming at a high speed can thus be improved. Moreover, the output information of the EVS 12 is sparse, so that a processing load can be reduced according to the embodiment.

Note that, in the embodiment, the integrated information processing/control unit 508 is not limited to performing processing of integrating pieces of output information from the EVS 12 and the image sensor 11. The pieces of output information can be individually performed.

The event preprocessing unit 514 can perform various pieces of processing on event data and a pixel signal acquired via the communication unit 502, and output the event data and the pixel signal to the event information processing unit 516 to be described later. The event data and the pixel signal are raw data transmitted from the EVS 12 of the camera head 5005. Examples of the processing include integration of pixel signals (generation of frame data (event image data)) for a certain period (frame length) from the EVS 12 and adjustment of the frame length. More specifically, the event preprocessing unit 514 includes a processor such as a CPU and a GPU. The processor operates in accordance with a predetermined program, whereby the above-described processing is performed.

The event information processing unit 516 can perform image processing based on the event data and the pixel signal processed by the event preprocessing unit 514. Furthermore, the event information processing unit 516 may recognize a surgical tool such as forceps, a specific living body site, a shape, bleeding, and the like by detecting the shape of an edge of a subject included in each surgical site image using various image recognition techniques.

The synchronization control unit 520 generates a synchronization control signal for synchronizing the camera head 5005 with the light source device 5043 based on a control signal from the integrated information processing/control unit 508, and controls the light source device 5043 via the communication unit 522. For example, the EVS 12 can make imaging preferable by adjusting the intensity of light applied from the light source device 5043 and a cycle of intensity change based on information on brightness of a screen based on an image from the image sensor 11.

The display information generation unit 530 displays an image of a surgical site on the display device 5041 based on a pixel signal processed by the integrated information processing/control unit 508. In the embodiment, the display information generation unit 530 may display, for example, segmentation information following the size, distance, and deformation of an organ on the display device 5041 in addition to the image of a surgical site.

### (Light Source Device 5043)

As illustrated in FIG. 5, the light source device 5043 mainly includes a communication unit 602, a light source control unit 604, and a light source unit 606. The communication unit 602 includes a communication device for transmitting and receiving various pieces of information (e.g., control signals) to and from the CCU 5039. The light source control unit 604 controls driving of the light source unit 606 based on a control signal from the CCU 5039 received via the communication unit 602. The light source unit 606 includes a light source such as an LED. The light source unit 606 supplies irradiation light to a surgical site under the control of the light source control unit 604.

### (Arm Control Device 5045)

As illustrated in FIG. 5, the arm control device 5045 mainly includes a communication unit 702, an arm trajectory generation unit 704, and an arm control unit 706. The communication unit 702 includes a communication device for transmitting and receiving various pieces of information (e.g., control signals) to and from the CCU 5039. The arm trajectory generation unit 704 can generate trajectory information serving as autonomous operation control information for causing the support arm portion 5027 to autonomously operate based on the control information from the CCU 5039. Then, the arm control unit 706 controls driving of an arm actuator 802 of the support arm portion 5027 based on the generated trajectory information. In the embodiment, sudden movement of a surgical tool, which is a subject, can also be detected in real time by the EVS 12 having high time resolution, so that the camera head 5005 can be instantaneously moved by the support arm portion 5027 so as not to interfere with the surgical tool. Therefore, surgery can be safely performed.

### 1.8 Operation Example

Next, an operation example of the endoscopic surgery system 5000 according to the embodiment will be described. In the embodiment, as described above, a deterioration in visibility/recognizability of a video due to reflection is inhibited by specifically removing only polarized light by utilizing polarization of light at the time when light is reflected at a highly reflective object such as a liquid surface.

### 1.8.1 Main Operation Example

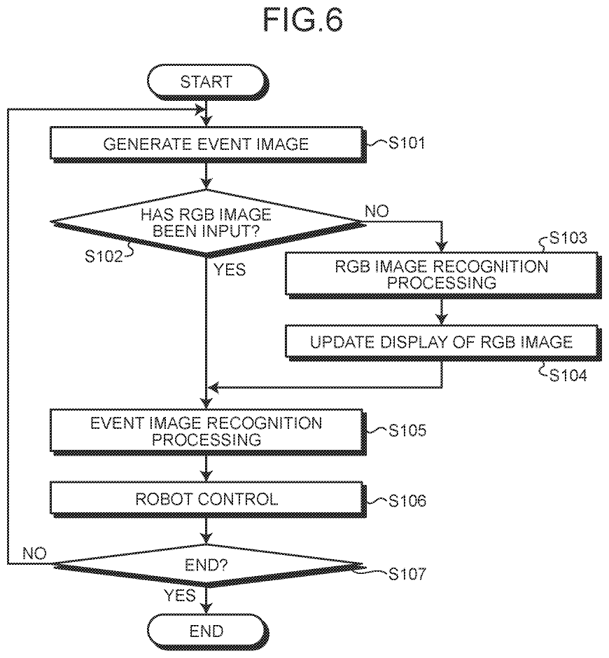

FIG. 6 is a flowchart illustrating one example of a main operation of the endoscopic surgery system according to the embodiment. Note that the following description is focused on an operation of each unit inside the CCU 5039.

As illustrated in FIG. 6, first, in Step S101, the event preprocessing unit 514 generates event image data (frame data) based on event data input from the EVS 12 during a predetermined frame period. Note that the event preprocessing unit 514 may generate the event image data at a frame rate higher than the frame rate of the image sensor 11, such as 1000 frames per second (fps). Real-time performance of measurement/recognition processing of an abdominal cavity environment can be improved by executing image recognition processing (S105) in a subsequent stage based on the event image data of a higher frame rate than that of the RGB image data.

Next, in Step S102, it is determined whether or not RGB image data has been input at a predetermined frame rate (e.g., 60 fps) from the image sensor 11. For example, the RGB signal/image processing unit 504 may execute the determination. When the RGB image data has not been input (NO in Step S102), the operation proceeds to Step S105.

In contrast, when the RGB image data has been input (YES in Step S102), the RGB recognition unit 506 executes recognition processing on the RGB image data processed by the RGB signal/image processing unit 504 (Step S103), and inputs the result together with the RGB image data to the integrated information processing/control unit 508. The integrated information processing/control unit 508 performs predetermined processing on the input RGB image data, and then inputs the processed RGB image data to the display information generation unit 530. In contrast, the display information generation unit 530 updates an RGB image displayed on the display device 5041 based on the input RGB image data (Step S104). This causes a video, which has been captured by the endoscope 5001 and subjected to predetermined processing, to be displayed on the display device 5041 substantially in real time. Thereafter, the operation proceeds to Step S105.

In Step S105, the event information processing unit 516 executes recognition processing on the event image data generated by the event preprocessing unit 514, and inputs the result to the integrated information processing/control unit 508 together with the RGB image data.

In Step S106, the integrated information processing/control unit 508 generates a control signal for causing the support arm portion 5027 to execute a desired operation based on a recognition result of the event image data input from the event information processing unit 516 (and, in some cases, recognition result of RGB image data input from the RGB recognition unit 506), and inputs the generated control signal to the arm control device 5045. This causes the support arm portion 5027 to execute a desired operation based on a control signal from the arm control device 5045.

Thereafter, in Step S107, for example, an overall control unit (not illustrated) and the like of the CCU 5039 determines whether or not to end the operation. When the operation is to be ended (YES in Step S107), the operation ends. In contrast, when the operation is not to be ended (NO in Step S107), the operation returns to Step S101, and the subsequent operations are executed.

### 1.8.2 Example of Polarizing Filter Adjustment Operation

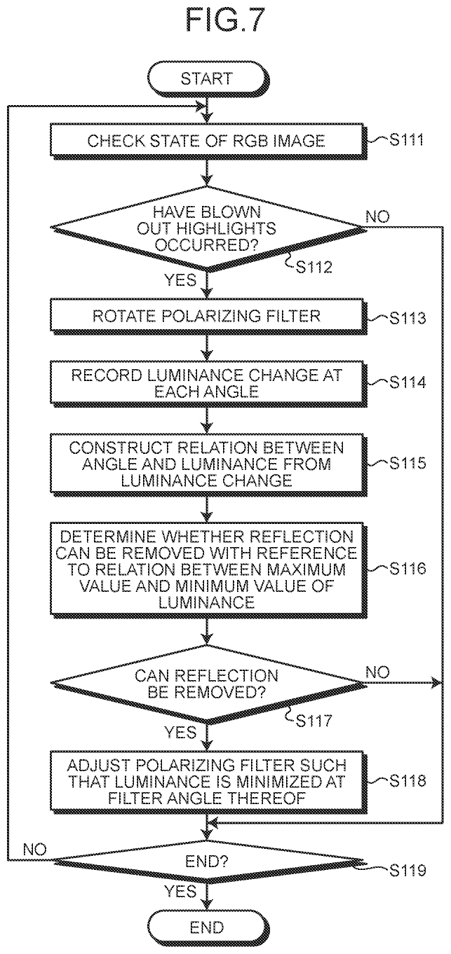

Furthermore, the endoscopic surgery system 5000 executes a polarizing filter adjustment operation for adjusting a rotation angle of the polarizing filter 15 in parallel with the above-described main operation. FIG. 7 is a flowchart illustrating one example of the polarizing filter adjustment operation according to the embodiment. Note that, although, in the following description, a case where the integrated information processing/control unit 508 in the CCU 5039 executes the polarizing filter adjustment operation will be exemplified, this is not a limitation. Another unit in the CCU 5039 may execute the polarizing filter adjustment operation.

As illustrated in FIG. 7, in the operation, the integrated information processing/control unit 508 first checks the state of the RGB image data input via the RGB recognition unit 506 (Step S111). The state of the RGB image data may be, for example, whether or not there is a pixel whose pixel value is saturated, that is, whether or not the image has blown out highlights. Note, however, that this is not a limitation. Whether or not sufficient contrast has been obtained may be checked.

When the RGB image data is in a normal state, that is, in the example, when blown out highlights have not occurred (NO in Step S112), the integrated information processing/control unit 508 proceeds to Step S119. In contrast, when blown out highlights have occurred (YES in Step S112), the integrated information processing/control unit 508 inputs an instruction to rotate the polarizing filter 15 around an optical axis to the adjustment mechanism 20 of the camera head 5005 via the communication units 502 and 102 (Step S113). The adjustment mechanism 20 may rotate the polarizing filter 15 by a predetermined angle, and the range thereof may be, for example, 360 degrees. Note, however, that this is not a limitation. The adjustment mechanism 20 may rotate the polarizing filter 15 at a constant speed. Furthermore, the polarizing filter 15 is only required to have a rotation range sufficient to construct the relation between an angle around the optical axis of the polarizing filter 15 and the luminance of the RGB image data acquired by the image sensor 11 in Step S115 to be described later.

During the rotation of the polarizing filter 15 in Step S113, the event data generated by the EVS 12 and the angle around the optical axis of the polarizing filter 15 (hereinafter, simply referred to as angle of polarizing filter 15) acquired by the encoder 22 of the adjustment mechanism 20 are input to the integrated information processing/control unit 508. Thus, the integrated information processing/control unit 508 records a luminance change for each pixel at each angle of the polarizing filter 15 in a memory (not illustrated) or the like based on the event data input for each angle of the polarizing filter 15 (Step S114). Note that the luminance change based on the event data may be, for example, the number of pieces of event data generated by the EVS 12 while the polarizing filter 15 is rotated by a predetermined angle.

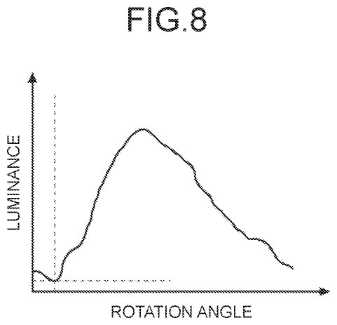

Next, the integrated information processing/control unit 508 constructs the relation between an angle around the optical axis of the polarizing filter 15 and luminance (hereinafter, simply referred to as relation between filter angle and luminance) from a luminance change at each angle of the polarizing filter 15 recorded in Step S114 (Step S115). FIG. 8 is a graph illustrating one example of the relation between the filter angle and the luminance constructed in Step S115.

Next, the integrated information processing/control unit 508 determines whether or not reflected light that causes saturation of the pixel value can be removed by rotating the polarizing filter 15 with reference to the relation between the maximum value and the minimum value of the luminance in the constructed relation (graph) based on the relation between the filter angle and the luminance constructed in Step S115 (Step S116). For example, when the difference between the maximum value and the minimum value of the luminance in the constructed relation (graph) is equal to or greater than a preset threshold, the integrated information processing/control unit 508 may determine that the reflected light can be removed by rotation of the polarizing filter 15.

When it is determined that the reflected light cannot be removed by rotation of the polarizing filter 15 (NO in Step S117), the integrated information processing/control unit 508 proceeds to Step S119. In contrast, when it is determined that the reflected light can be removed by rotation of the polarizing filter 15 (YES in Step S117), the integrated information processing/control unit 508 inputs an instruction for adjusting the angle around the optical axis of the polarizing filter 15 such that the luminance is minimized at the angle to the adjustment mechanism 20 of the camera head 5005 via the communication units 502 and 102 based on the relation (graph) constructed in Step S115, for example (Step S118).

In Step S119, the integrated information processing/control unit 508 determines whether or not to end the operation. When the operation is to be ended (YES in Step S119), the integrated information processing/control unit 508 ends the operation. In contrast, when the operation is not to be ended (NO in Step S119), the integrated information processing/control unit 508 returns to Step S111, and executes subsequent operations.

As described above, the integrated information processing/control unit 508 functions as an adjustment unit that adjusts an amount of light transmitted through the polarizing filter 15 based on an amount of the reflected light L2 incident on the EVS 12 (and image sensor 11). The adjustment unit may include the adjustment mechanism 20 in the camera head 5005. Saturation of the pixel values of the RGB image data acquired by the image sensor 11 and event image data based on the event data generated by the EVS 12, that is, occurrence of blown out highlights can be inhibited by the adjustment unit adjusting the angle around the optical axis of the polarizing filter 15 such that the luminance is minimized at the angle. This can inhibit a deterioration in robustness in image recognition.

### 1.9 Actions/Effects

As described above, for example, the real-time performance at the time when an abdominal cavity environment is measured/recognized from an image captured by using an endoscope depends on the frame rate of an imaging device mounted in the endoscope. A common imaging device of a rame rate of approximately 60 fps is usually used for the endoscope, so that there is room for improving the real-time performance. According to the embodiment, an image is recognized based on event image data of a higher frame rate than that of RGB image data, which can improve the real-time performance of measurement/recognition processing of the abdominal cavity environment. Furthermore, according to the embodiment, saturation of the pixel values of the RGB image data acquired by the image sensor 11 and event image data based on the event data generated by the EVS 12, that is, occurrence of blown out highlights can be inhibited by adjusting the angle around the optical axis of the polarizing filter 15 such that the luminance is minimized at the angle, so that a deterioration in robustness in image recognition can be inhibited.

### 1.10 Variations

Subsequently, variations of the above-described embodiment will be described. A variation of the polarizing filter adjustment operation described with reference to FIG. 7 and a variation of the adjustment mechanism 20 in the optical system 400 of the endoscope 5001 described with reference to FIG. 2 will be described below.

### 1.10.1 Variations of Polarizing Filter Adjustment Operation

First, variations of the polarizing filter adjustment operation will be described in some examples.

### 1.10.1.1 First Variation

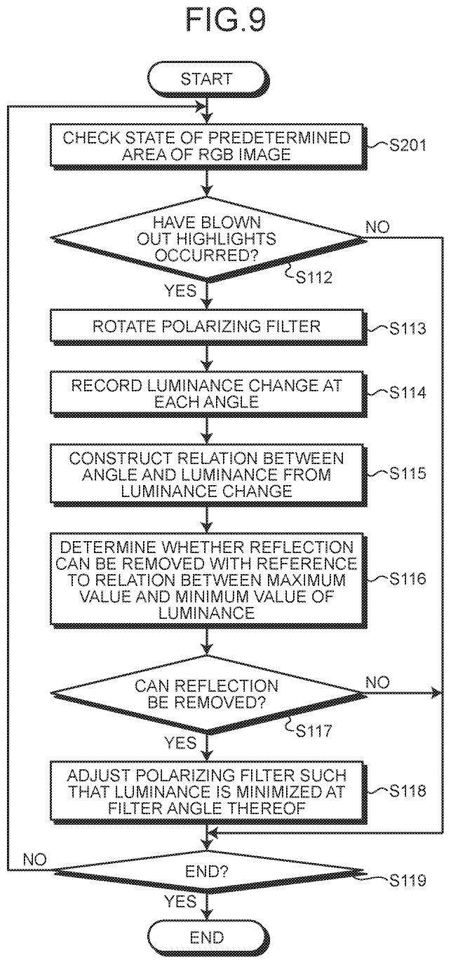

FIG. 9 is a flowchart illustrating a first variation of the polarizing filter adjustment operation according to the embodiment. As illustrated in FIG. 9, in the polarizing filter adjustment operation according to the first variation, Step S111 in FIG. 7 is replaced with Step S201 in an operation similar to the polarizing filter adjustment operation described with reference to FIG. 7.

As illustrated in FIG. 9, in Step S201, the integrated information processing/control unit 508 checks the state of a predetermined area in the RGB image data input via the RGB recognition unit 506. The predetermined area may be, for example, a central area of the RGB image data. Note, however, that this is not a limitation. When the endoscopic surgery system 5000 includes a sensor that detects a direction of line of sight of a surgeon and the like, for example, the predetermined area may be set based on which region in a video displayed on the display device 5041 the surgeon and the like look at. Furthermore, for example, when the integrated information processing/control unit 508 acquires a position in an image of a surgical tool or a specific site of the surgical tool such as an electric scalpel and a distal end of the electric scalpel as a result of recognition processing on the event image data and/or the RGB image data, the predetermined area may be set based on the acquired position.

A determination area for the RGB image data is narrowed down to a central area, an area based on a line of sight, or an area based on the position and the like of a surgical tool as described above, which enables omission of determination processing on the other areas with low importance. Whether or not blown out highlights have occurred can thereby be more quickly determined, and the real-time performance of measurement/recognition processing of the abdominal cavity environment can be further improved. Other operations may be similar to those of the above-described embodiment, so that detailed description thereof is omitted here.

### 1.10.1.2 Second Variation

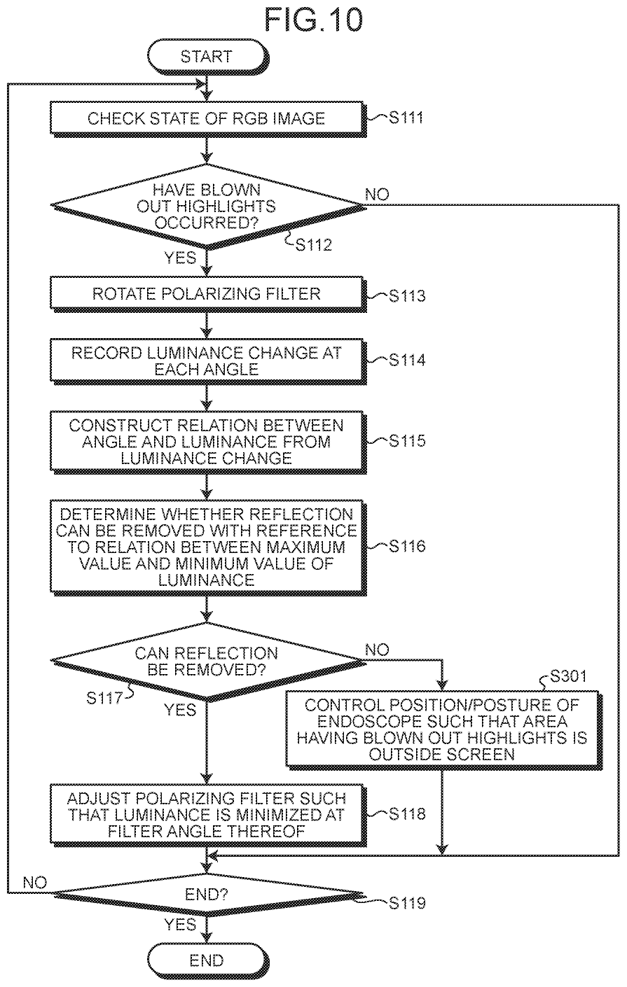

FIG. 10 is a flowchart illustrating a second variation of the polarizing filter adjustment operation according to the embodiment. As illustrated in FIG. 10, the polarizing filter adjustment operation according to the second variation is configured such that Step S301 is executed in a case where it is determined that reflected light cannot be removed by rotation of the polarizing filter 15 in Step S116 in FIG. 7 (NO in Step S117) in an operation similar to the polarizing filter adjustment operation described with reference to FIG. 7.

As illustrated in FIG. 10, in Step S301, the integrated information processing/control unit 508 inputs an instruction for controlling the position and posture of the endoscope 5001 to the arm trajectory generation unit 704 of the arm control device 5045 via the communication units 532 and 702 such that the area, where blown out highlights have occurred, specified in Step S111 is outside a screen of the RGB image data (and event image data), that is, an area in real space corresponding to the specified area where blown out highlights have occurred is outside angles of view of the image sensor 11 and the EVS 12. In contrast, the arm trajectory generation unit 704 generates trajectory data for moving the endoscope 5001 to a position and posture at which the area in real space corresponding to the specified area where blown out highlights have occurred is outside the angles of view of the image sensor 11 and the EVS 12, and inputs the trajectory data to the arm control unit 706. The arm control unit 706 generates a control signal for controlling the arm actuator 802 of the support arm portion 5027 based on the trajectory data input from the arm trajectory generation unit 704, and inputs the control signal to the arm actuator 802. This causes the position and posture of the endoscope 5001 to be controlled such that the area in real space corresponding to the specified area where blown out highlights have occurred is outside the angles of view of the image sensor 11 and the EVS 12.

As described above, in the second variation, when it is determined that reflected light cannot be removed by rotation of the polarizing filter 15 in Step S116 (NO in Step S117), the position and posture of the endoscope 5001 is controlled such that the area in real space corresponding to the area where blown out highlights have occurred is outside the angles of view of the image sensor 11 and the EVS 12. In other words, the integrated information processing/control unit 508 determines whether or not the amount of the reflected light L2 transmitted through the polarizing filter 15 can be adjusted based on the relation between the angle around the optical axis of the polarizing filter 15 and the luminance of the reflected light L2. When it is determined that the adjustment is impossible, the integrated information processing/control unit 508 causes the arm control device 5045 to control the support arm portion 5027 to adjust the amount of light incident on the EVS 12 and the image sensor 11. This can reduce blown out highlights in an image without depending on the polarizing filter 15, so that the deterioration in robustness in image recognition can be inhibited. Other operations may be similar to those of the above-described embodiment, so that detailed description thereof is omitted here.

### 1.10.2 Variations of Adjustment Mechanism

First, variations of the adjustment mechanism 20 will be described in some examples.

### 1.10.2.1 First Variation

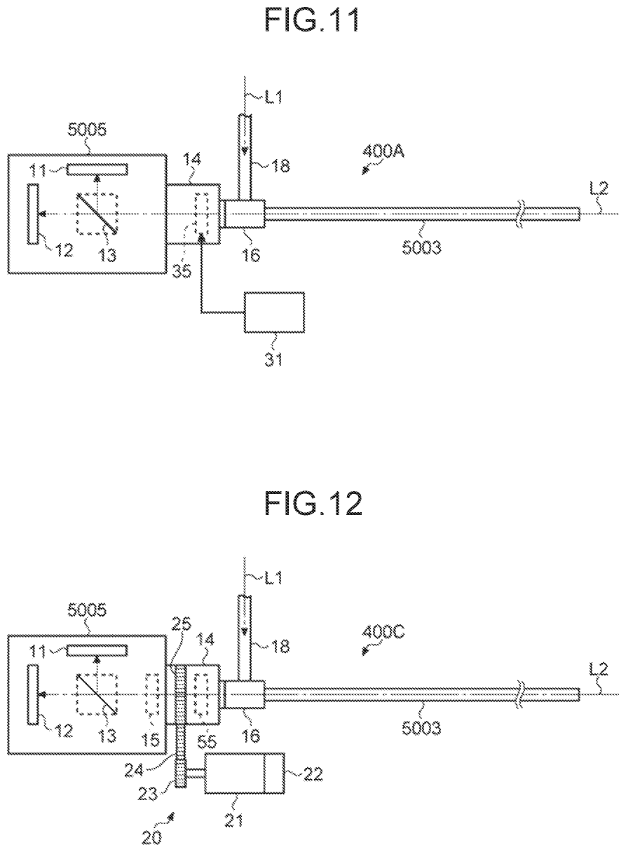

Although, in the above-described embodiment, a case where a linear polarizer having a function of the linear polarizer itself selectively transmitting, as a physical property thereof, a linearly polarized component in a specific direction, such as a polarizing prism and a polarizing plate, is used as the polarizing filter 15 has been exemplified, this is not a limitation. For example, as in an optical system 400A in FIG. 11, a wire grid polarizer and the like capable of adjusting the polarization direction of the linearly polarized component to be selectively transmitted in accordance with an electric field formed by a voltage supplied from a power supply 31 may be used as a polarizing filter 35.

### 1.10.2.2 Second Variation

Furthermore, although, in the above-described embodiment and the first variation of the adjustment mechanism, a case where an amount of light incident on the camera head 5005 is adjusted by adjusting the polarization direction of the linearly polarized component to be selectively transmitted with respect to the reflected light L2 has been exemplified, this is not a limitation. For example, as in an optical system 400C in FIG. 12, a polarization rotator 55 may be fixed in the joint portion 14 capable of rotating to the camera head 5005. The polarization rotator 55 rotates the polarization direction of incident light. For example, a Faraday rotator having a magneto-optical effect for rotating a polarization state of light by a magnetic field may be used as the polarization rotator 55.

In the second variation, the polarizing filter 15 may be fixed around the optical axis of the reflected light L2 (e.g., fixed to camera head 5005). Furthermore, the polarizing filter 15 may be located at any position between the polarization rotator 55 and the beam splitter 13, such as in the joint portion 14 and in the camera head 5005.

In such configuration, the polarization direction of the reflected light L2 incident on the polarizing filter 15 can be adjusted by rotating the polarization rotator 55 around the optical axis of the reflected light L2. This enables adjustment of an amount of light incident on the image sensor 11 and the EVS 12 transmitted through the polarizing filter 15 similarly to the above-described embodiment and the first variation of the adjustment mechanism.

### 1.10.2.3 Third Variation

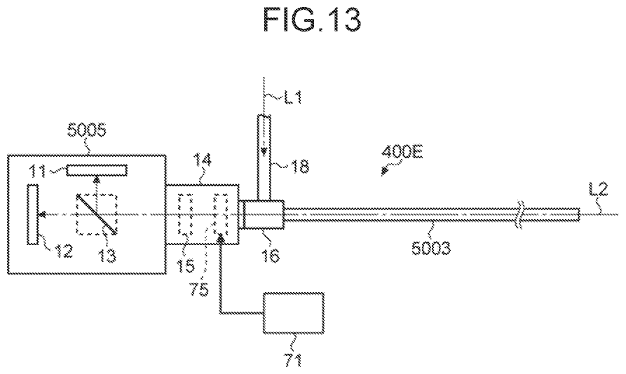

Although, in the above-described second variation, a case where a Faraday rotator having a magneto-optical effect or the like is used as the polarization rotator 55 has been exemplified, this is not a limitation. For example, as in an optical system 400E in FIG. 13, a polarization rotator using a magneto-optical effect (Faraday effect) for adjusting the polarization direction of transmitted light in accordance with a magnetic field generated by a voltage supplied from a power supply 71 may be used as a polarization rotator 75.

### 2. Second Embodiment

By the way, the EVS 12 cannot detect an event unless there is a change in luminance equal to or greater than a predetermined threshold. Therefore, the EVS 12 cannot capture a subject having no or little luminance change. Thus, in the second embodiment, a drive unit that moves the EVS 12 in a direction perpendicular to the optical axis of the reflected light L2 reflected by the beam splitter 13 may be provided, and a change in luminance may be forcibly generated by moving the EVS 12 by using the drive unit, thereby prompting detection of an event of the EVS 12.

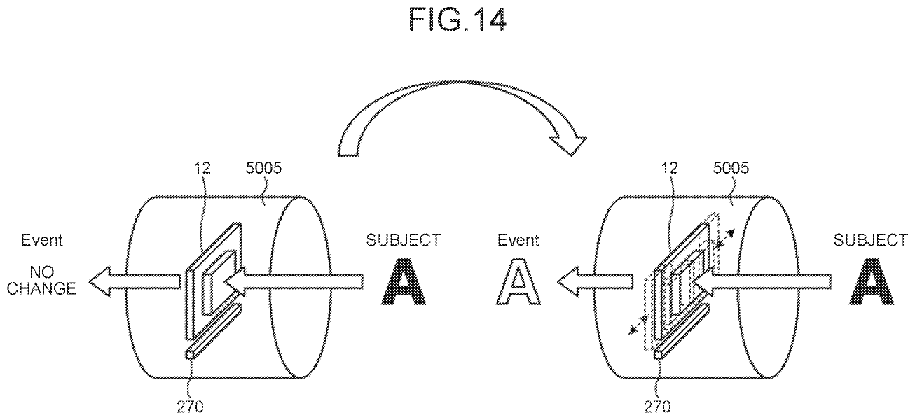

FIG. 14 is a schematic diagram illustrating one example of the drive unit that moves the EVS according to the embodiment. As illustrated in FIG. 14, in the second embodiment, an actuator (drive unit) 270 is provided as a drive unit that moves the EVS 12 itself along a predetermined direction.

As illustrated on the left side of FIG. 14, when a subject does not move, luminance is not changed, and an event does not occur, so that the EVS 12 cannot capture an image of the subject. Thus, in the embodiment, in order to forcibly capture the image of the subject with the EVS 12, the actuator 270 slightly moves the EVS 12 in a right and left direction as illustrated on the right side of FIG. 14. When the EVS 12 itself is slightly moved by the actuator 270, the EVS 12 detects movement of an image formed on a light receiving surface as a luminance change. This enables an image of a subject that does not move to be captured. Note that the actuator 270 may slightly move the EVS 12 not only in the right and left direction but in a vertical direction. Furthermore, the actuator 270 may move not only the EVS 12 but the optical system 400 in a direction perpendicular to an optical axis.

Moreover, in the embodiment, when the EVS 12 cannot detect the subject, the intensity of light may be changed for a short period (light irradiation cycle may be changed) such that a luminance change is increased by cooperation with a light source device 600. Moreover, in the embodiment, when the EVS 12 cannot detect the subject, a threshold to be compared with a luminance change amount may be dynamically changed.

As described above, according to the embodiment, the EVS 12 can capture even a subject having no or little luminance change.

Other configurations, operations, and effects may be similar to those of the above-described embodiment or the variations thereof, so that detailed description thereof is omitted here.

### 3. Hardware Configuration

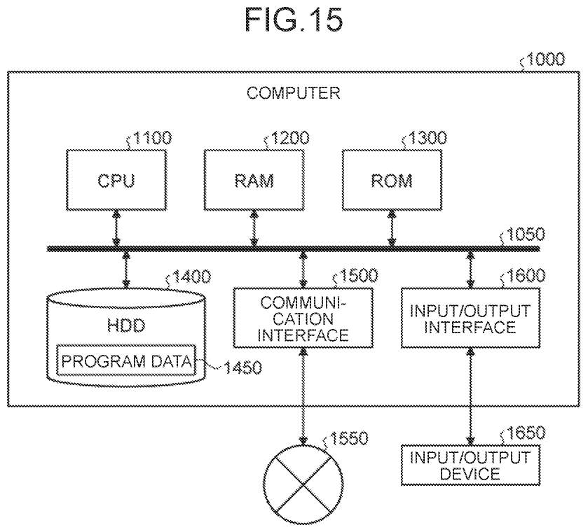

The CCU 5039, the arm control device 5045, the treatment tool control device 5049, and the like according to the above-described embodiments and the variations and application examples thereof can be achieved by, for example, a computer 1000 having a configuration as illustrated in FIG. 15. FIG. 15 is a hardware configuration diagram illustrating one example of the computer 1000 that implements the respective functions of the CCU 5039, the arm control device 5045, the treatment tool control device 5049, and the like. The computer 1000 includes a CPU 1100, a RAM 1200, a read only memory (ROM) 1300, a hard disk drive (HDD) 1400, a communication interface 1500, and an input/output interface 1600. Units of the computer 1000 are connected by a bus 1050.

The CPU 1100 operates based on a program stored in a ROM 1300 or an HDD 1400, and controls each unit. For example, the CPU 1100 develops the program stored in the ROM 1300 or the HDD 1400 on the RAM 1200, and executes processing corresponding to various programs.

The ROM 1300 stores a boot program of a basic input output system (BIOS) and the like executed by the CPU 1100 at the time when the computer 1000 is started, a program depending on hardware of the computer 1000, and the like.

The HDD 1400 is a computer-readable recording medium that non-transiently records a program executed by the CPU 1100, data used by the program, and the like. Specifically, the HDD 1400 is a recording medium that records a program for implementing each operation according to the present disclosure. The program is one example of program data 1450.

The communication interface 1500 connects the computer 1000 with an external network 1550 (e.g., Internet). For example, the CPU 1100 receives data from another device, and transmits data generated by the CPU 1100 to another device via the communication interface 1500.

The input/output interface 1600 connects an input/output device 1650 with the computer 1000. For example, the CPU 1100 receives data from an input device such as a keyboard and a mouse via the input/output interface 1600. Furthermore, the CPU 1100 transmits data to an output device such as a display, a speaker, and a printer via the input/output interface 1600. Furthermore, the input/output interface 1600 may function as a medium interface that reads a program and the like recorded in a predetermined recording medium. The medium includes, for example, an optical recording medium such as a digital versatile disc (DVD) and a phase change rewritable disk (PD), a magneto-optical recording medium such as a magneto-optical disk (MO), a tape medium, a magnetic recording medium, a semiconductor memory, and the like.

For example, when the computer 1000 functions as each of the CCU 5039, the arm control device 5045, the treatment tool control device 5049, and the like according to the above-described embodiment, the CPU 1100 of the computer 1000 implements a function of each of the CCU 5039, the arm control device 5045, the treatment tool control device 5049, and the like by executing the program loaded on the RAM 1200. Furthermore, the HDD 1400 stores a program and the like according to the present disclosure. Note that the CPU 1100 reads the program data 1450 from the HDD 1400 and executes the program data 1450. In another example, the CPU 1100 may acquire these programs from another device via the external network 1550.

Although the embodiments of the present disclosure have been described above, the technical scope of the present disclosure is not limited to the above-described embodiments as it is, and various modifications can be made without departing from the gist of the present disclosure. Furthermore, components of different embodiments and variations may be appropriately combined.

Furthermore, the effects in each embodiment described in the present specification are merely examples and not limitations. Other effects may be exhibited.

Note that the present technology can also have the configurations as follows.

(1)

A medical observation system including:
a first imaging unit that includes a plurality of pixels each of which detects a change in luminance of incident light as an event and that acquires an image of an environment in an abdominal cavity of a living body;
a polarizing filter disposed on an optical path of the incident light incident on the first imaging unit; and
an adjustment unit that adjusts luminance of the incident light incident on the first imaging unit by adjusting luminance of light transmitted through the polarizing filter based on the image acquired by the first imaging unit.
   (2)

The medical observation system according to (1),
wherein the polarizing filter is a linear polarizer that selectively transmits a linearly polarized component.
   (3)

The medical observation system according to (2),
wherein the adjustment unit adjusts luminance of light transmitted through the polarizing filter by rotating the polarizing filter around an optical axis of the incident light.
   (4)

The medical observation system according to (2),
wherein the polarizing filter is a linear polarizer that changes a polarization direction of transmitted light in accordance with an applied voltage, and the adjustment unit adjusts luminance of light transmitted through the polarizing filter by adjusting a voltage to be applied to the polarizing filter.
   (5)

The medical observation system according to (2), further including
a rotator disposed on a side opposite to the first imaging unit across the polarizing filter on an optical path of the incident light,
wherein the adjustment unit adjusts luminance of light transmitted through the polarizing filter by rotating the rotator around an optical axis of the incident light.
   (6)

The medical observation system according to (2), further including
a rotator disposed on a side opposite to the first imaging unit across the polarizing filter on an optical path of the incident light,
wherein the adjustment unit adjusts luminance of light transmitted through the polarizing filter by adjusting a voltage to be applied to the rotator.
   (7)

The medical observation system according to any one of (1) to (6),
wherein the adjustment unit constructs relation between an angle around the optical axis of the polarizing filter and luminance of the incident light based on a change in luminance of the incident light incident on the first imaging unit at a time when the polarizing filter is rotated around an optical axis of the incident light, and adjusts luminance of light transmitted through the polarizing filter such that luminance of the incident light incident on the first imaging unit is minimized based on the relation that has been constructed.
   (8)

The medical observation system according to (7), further including:
an arm portion that supports the first imaging unit; and
an arm control device that controls the arm portion,
wherein the adjustment unit determines whether or not luminance of the incident light incident on the first imaging unit is allowed to be adjusted based on the relation between an angle around the optical axis of the polarizing filter and luminance of the incident light, and, when determining that adjustment is impossible, adjusts luminance of light transmitted through the polarizing filter by causing the arm control device to control the arm portion.
   (9)

The medical observation system according to (8),
wherein the adjustment unit determines whether or not luminance of the incident light incident on the first imaging unit is allowed to be adjusted based on difference between maximum luminance and minimum luminance of the incident light incident on the first imaging unit at a time when the polarizing filter is rotated around the optical axis of the incident light.
   (10)

The medical observation system according to any one of (1) to (9), further including
a light source device that illuminates the abdominal cavity.
   (11)

The medical observation system according to any one of (1) to (10), further including:
a second imaging unit that acquires an image of an environment in the abdominal cavity; and
a beam splitter that is disposed between the polarizing filter and the first imaging unit on an optical path of the incident light and that splits light transmitted through the polarizing filter into light incident on the first imaging unit and light incident on the second imaging unit.
   (12)

The medical observation system according to (11), further including
a display device that displays the image acquired by the second imaging unit.
   (13)

The medical observation system according to any one of (1) to (12), further including
a drive unit that moves the first imaging unit in a direction perpendicular to an optical axis of the incident light.
   (14)

An information processing apparatus including a program causing a computer to execute processing of adjusting luminance of incident light incident on an imaging unit including a plurality of pixels each of which detects a change in luminance of incident light as an event,
wherein the program causes the computer to execute processing of adjusting luminance of the incident light incident on the imaging unit by adjusting luminance of light transmitted through a polarizing filter disposed on an optical path of incident light incident on the imaging unit based on an image of an environment in an abdominal cavity of a living body acquired by the imaging unit.
   (15)

An information processing method including
adjusting luminance of incident light incident on the imaging unit by adjusting luminance of light transmitted through a polarizing filter disposed on an optical path of incident light incident on the imaging unit based on an image of an environment in an abdominal cavity of a living body acquired by an imaging unit including a plurality of pixels each of which detects a change in luminance of incident light as an event.

### REFERENCE SIGNS LIST

11 IMAGE SENSOR
12 EVS
13 BEAM SPLITTER
14 JOINT PORTION
15, 35 POLARIZING FILTER
16 MERGING PORTION
18 OPTICAL FIBER CABLE
20 ADJUSTMENT MECHANISM
21 DRIVE UNIT
22 ENCODER
23, 24, 25 GEAR
55, 75 POLARIZATION ROTATOR
71 POWER SUPPLY
102, 502, 522, 532, 602, 702 COMMUNICATION UNIT
270 ACTUATOR
310 PERIPHERAL CIRCUIT UNIT
316 DRIVE CONTROL UNIT
504 RGB SIGNAL/IMAGE PROCESSING UNIT
506 RGB RECOGNITION UNIT
508 INTEGRATED INFORMATION PROCESSING/CONTROL UNIT
514 EVENT PREPROCESSING UNIT
516 EVENT INFORMATION PROCESSING UNIT
520 SYNCHRONIZATION CONTROL UNIT
530 DISPLAY INFORMATION GENERATION UNIT
604 LIGHT SOURCE CONTROL UNIT
606 LIGHT SOURCE UNIT
704 ARM TRAJECTORY GENERATION UNIT
706 ARM CONTROL UNIT
802 ARM ACTUATOR
400, 400A, 400C, 400E OPTICAL SYSTEM
410 OPTICAL UNIT
5000 ENDOSCOPIC SURGERY SYSTEM
5001 ENDOSCOPE
5003 LENS BARREL
5005 CAMERA HEAD
5027 SUPPORT ARM PORTION
5031 ARM PORTION
5039 CCU
5041 DISPLAY DEVICE
5043 LIGHT SOURCE DEVICE
5045 ARM CONTROL DEVICE

1. A medical observation system including:
a first imaging unit that includes a plurality of pixels each of which detects a change in luminance of incident light as an event and that acquires an image of an environment in an abdominal cavity of a living body;
a polarizing filter disposed on an optical path of the incident light incident on the first imaging unit; and
an adjustment unit that adjusts luminance of the incident light incident on the first imaging unit by adjusting luminance of light transmitted through the polarizing filter based on the image acquired by the first imaging unit.

2. The medical observation system according to claim 1,
wherein the polarizing filter is a linear polarizer that selectively transmits a linearly polarized component.

3. The medical observation system according to claim 2,
wherein the adjustment unit adjusts luminance of light transmitted through the polarizing filter by rotating the polarizing filter around an optical axis of the incident light.

4. The medical observation system according to claim 2,
wherein the polarizing filter is a linear polarizer that changes a polarization direction of transmitted light in accordance with an applied voltage, and
the adjustment unit adjusts luminance of light transmitted through the polarizing filter by adjusting a voltage to be applied to the polarizing filter.

5. The medical observation system according to claim 2, further including
a rotator disposed on a side opposite to the first imaging unit across the polarizing filter on an optical path of the incident light,
wherein the adjustment unit adjusts luminance of light transmitted through the polarizing filter by rotating the rotator around an optical axis of the incident light.

6. The medical observation system according to claim 2, further including
a rotator disposed on a side opposite to the first imaging unit across the polarizing filter on an optical path of the incident light,
wherein the adjustment unit adjusts luminance of light transmitted through the polarizing filter by adjusting a voltage to be applied to the rotator.

7. The medical observation system according to claim 1,
wherein the adjustment unit constructs relation between an angle around the optical axis of the polarizing filter and luminance of the incident light based on a change in luminance of the incident light incident on the first imaging unit at a time when the polarizing filter is rotated around an optical axis of the incident light, and adjusts luminance of light transmitted through the polarizing filter such that luminance of the incident light incident on the first imaging unit is minimized based on the relation that has been constructed.

8. The medical observation system according to claim 7, further including:
an arm portion that supports the first imaging unit; and
an arm control device that controls the arm portion,
wherein the adjustment unit determines whether or not luminance of the incident light incident on the first imaging unit is allowed to be adjusted based on the relation between an angle around the optical axis of the polarizing filter and luminance of the incident light, and, when determining that adjustment is impossible, adjusts luminance of light transmitted through the polarizing filter by causing the arm control device to control the arm portion.

9. The medical observation system according to claim 8,
wherein the adjustment unit determines whether or not luminance of the incident light incident on the first imaging unit is allowed to be adjusted based on difference between maximum luminance and minimum luminance of the incident light incident on the first imaging unit at a time when the polarizing filter is rotated around the optical axis of the incident light.

10. The medical observation system according to claim 1, further including
a light source device that illuminates the abdominal cavity.

11. The medical observation system according to claim 1, further including:
a second imaging unit that acquires an image of an environment in the abdominal cavity; and
a beam splitter that is disposed between the polarizing filter and the first imaging unit on an optical path of the incident light and that splits light transmitted through the polarizing filter into light incident on the first imaging unit and light incident on the second imaging unit.

12. The medical observation system according to claim 11, further including
a display device that displays the image acquired by the second imaging unit.

13. The medical observation system according to claim 1, further including
a drive unit that moves the first imaging unit in a direction perpendicular to an optical axis of the incident light.

14. An information processing apparatus including a program causing a computer to execute processing of adjusting luminance of incident light incident on an imaging unit including a plurality of pixels each of which detects a change in luminance of incident light as an event,
wherein the program causes the computer to execute processing of adjusting luminance of the incident light incident on the imaging unit by adjusting luminance of light transmitted through a polarizing filter disposed on an optical path of incident light incident on the imaging unit based on an image of an environment in an abdominal cavity of a living body acquired by the imaging unit.

15. An information processing method including
adjusting luminance of incident light incident on the imaging unit by adjusting luminance of light transmitted through a polarizing filter disposed on an optical path of incident light incident on the imaging unit based on an image of an environment in an abdominal cavity of a living body acquired by an imaging unit including a plurality of pixels each of which detects a change in luminance of incident light as an event.
